(19) United States
(12) Patent Application Publication
(10) Pub. No.: US 2017/0035824 A1
(43) Pub. Date: Feb. 9, 2017

(54) USAGE OF OBLONGIFOLIN C, A NATURAL COMPOUND FROM GARCINIA YUNNANENSIS HU, ON TREATING CANCER AS METASTASIS INHIBITOR AND AUTOPHAGIC FLUX INHIBITOR

(71) Applicant: Hong Kong Baptist University, Hong Kong (HK)

(72) Inventors: Hongxi XU, Shanghai (CN); Yuanzhi LAO, Shanghai (CN); Hongsheng TAN, Shanghai (CN); Naihan XU, Shanghai (CN); Xiaoyu WANG, Shanghai (CN); Kaixian CHEN, Shanghai (CN); Zhaoxiang BIAN, Hong Kong (HK); Dajian YANG, Chongqing (CN); Shilin CHEN, Beijing (CN); Aiping LU, Hong Kong (HK); Albert Sun Chi CHAN, Hong Kong (HK)

(21) Appl. No.: 15/297,165

(22) Filed: Oct. 19, 2016

Related U.S. Application Data
(63) Continuation-in-part of application No. 14/151,792, filed on Jan. 9, 2014.
(60) Provisional application No. 61/750,870, filed on Jan. 10, 2013.

(30) Foreign Application Priority Data
Mar. 26, 2013 (CN) 201310101235.5

(51) Int. Cl. A61K 36/38; A61K 31/122

(52) U.S. Cl. CPC A61K 36/38; A61K 31/122

(57) ABSTRACT

Disclosed is a natural compound, Oblongifolin C, isolated from a natural plant comprising Garcinia species such as Garcinia yunnanesis Hu, for its effects of anti-migration and anti-invasion against cancer and its use as an anticancer drug. The treated cancers comprise cervical cancers and esophageal cancers. The cancer treatment comprises inhibition of cancer metastasis and inhibition of autophagic flux.

### CROSS-REFERENCE TO RELATED APPLICATIONS

This application is a Continuation-in-Part Application of U.S. patent application Ser. No. 14/151,792 filed on Jan. 9, 2014 which claims the benefit of U.S. Provisional Patent Application Ser. No. 61/750,870 filed Jan. 10, 2013 and of Chinese Patent Application Serial Number 201310101235.5 filed Mar. 26, 2013. All of the foregoing applications are hereby incorporated by reference in their entirety.

### FIELD OF INVENTION

The present invention relates to a chemical entity isolated from natural sources for its therapeutic uses. More particularly, it relates to a compound that is naturally occurring in the plant of Garcinia yunnanensis Hu and its biological activity of antitumor effects.

### BACKGROUND OF INVENTION

Esophageal cancer is one of the most common malignancies and is associated with a dismal prognosis. Although treatment options have increased for some patients, overall progress has been modest. The emergence of disseminated metastasis remains the primary cause of mortality in cancer patients. Thus, there is a great need to develop more effective new treatments.

Citation or identification of any reference in this section or any other section of this application shall not be construed as an admission that such reference is available as prior art for the present application.

### SUMMARY OF INVENTION

Accordingly, it is an object of the present invention to provide a natural compound, Oblongifolin C, isolated from Garcinia yunnanensis Hu, for its effects of anti-migration and anti-invasion against cancer and its use as an anticancer drug.

In accordance with one aspect of the present invention, there is provided a composition for treating cancer comprising a compound with the chemical structure of

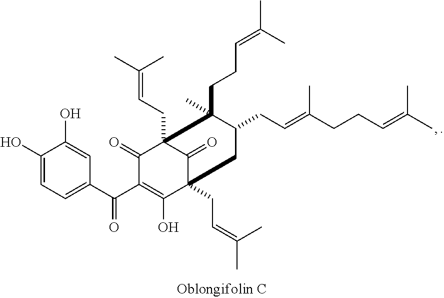 [SMILES: CC(C)=CCC/C(C)=C/C[C@@H]1C[C@]2(CC=C(C)C)C(=O)[C@@](CC=C(C)C)(C(=O)C(C(=O)c3ccc(O)c(O)c3)=C2O)[C@@]1(C)CCC=C(C)C]

In a first embodiment of one aspect of the present invention, the compound is isolated from a natural plant comprising Garcinia species such as Garcinia yunnanesis Hu.

In a second embodiment of one aspect of the present invention, the treated cancers comprise cervical cancers and esophageal cancers.

In a third embodiment of one aspect of the present invention, the cancer treatment comprises inhibition of cancer metastasis and inhibition of autophagic flux.

In a second aspect of the present invention, there is provided the use of Oblongifolin C in the development of anticancer drugs.

In one embodiment of the second aspect of the present invention, Oblongifolin C is used as an autophagy inhibitor.

In a third aspect of the present invention, there is provided a method of treating cancer comprises administering to a subject in need thereof a composition comprising an effective amount of Oblongifolin C.

In one embodiment of the third aspect of the present invention, Oblongifolin C is administered by intratumoral injection.

In one embodiment of the third aspect of the present invention, the subject in need thereof is a human.

In one embodiment of the third aspect of the present invention, the effective amount of Oblongifolin C is 2.43 mg Oblongifolin per 50 kg of a human.

In one embodiment of the third aspect of the present invention, said composition is administered to the subject in need thereof once every two days for two weeks.

Those skilled in the art will appreciate that the invention described herein is susceptible to variations and modifications other than those specifically described.

The invention includes all such variation and modifications. The invention also includes all of the steps and features referred to or indicated in the specification, individually or collectively, and any and all combinations or any two or more of the steps or features.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

Other definitions for selected terms used herein may be found within the detailed description of the invention and apply throughout. Unless otherwise defined, all other technical terms used herein have the same meaning as commonly understood to one of ordinary skill in the art to which the invention belongs.

Other aspects and advantages of the invention will be apparent to those skilled in the art from a review of the ensuing description.

### BRIEF DESCRIPTION OF DRAWINGS

The above and other objects and features of the present invention will become apparent from the following description of the invention, when taken in conjunction with the accompanying drawings, in which:

FIG. 1 shows the structure of Oblongifolin C.

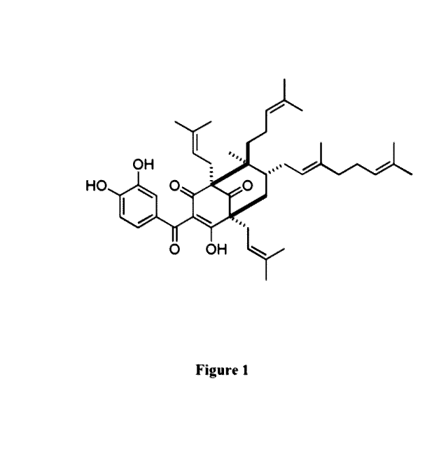

FIG. 2 shows the wound healing assay of Oblongifolin C in ECA109 cells.

FIG. 3 shows the transwell assay of Oblongifolin C in ECA109 cells. FIG. 3(a) shows the statistical assay of migration cells in different Oblongifolin C concentration treatment. FIG. 3(b) shows the Giemsa staining of migration cells in different Oblongifolin C concentration treatment.

FIG. 4 shows the matrigel assay of Oblongifolin C in ECA109 cells. FIG. 4(a) shows the statistical assay of invasion cells in different Oblongifolin C concentration treatment. FIG. 4(b) shows the Giemsa staining of invasion cells in different Oblongifolin C concentration treatment.

FIG. 5 shows the HeLa-LC3-GFP cells treated with OC (5, 10 and 20 μM) for 24 hours and the images analyzed by fluorescence microscopy.

FIG. 6 shows the percentage of GFP-LC3 positive cells.

FIG. 7 shows the HepG2, CNE, HCT116, MCF7 and MEF cells treated with OC (10 11M) for 24 hours and the images analyzed by fluorescence microscopy.

FIG. 8 shows the HeLa or MEF cells treated with OC (2, 5, 10 and 25 μM) for 24 hours that were analyzed by western blotting for endogenous LC3. GAPDH was used as a loading control. An image J densitometric analysis of the LC3-II/GAPDH ratio from LC3 immunoblots was shown.

FIG. 9 shows the HeLa or MEF cells treated with OC (15 μM) for 0, 4, 8, 12 and 24 hours that were analyzed by western blotting for endogenous LC3. GAPDH was used as a loading control. An image J densitometric analysis of the LC3-II/GAPDH ratio from LC3 immunoblots was shown.

FIG. 10 shows the HeLa cells or MEFs treated with OC (15 μM) over a certain time period. Samples were analyzed by western blotting for endogenous SQSTM1 and GAPDH. An image J densitometric analysis of the SQSTM1/GAPDH ratio from immunoblots was shown.

FIG. 11 shows the HeLa cells stably expressing GFP-LC3 treated with OC (15 μM), HCQ (50 μM) for 8 hours, or cultured in EBSS solution for 2 hours as indicated. Live cell images of GFP-LC3 and LysoTracker Red were taken using Olympus confocal microscope. The enlarged images show the colocalization of two signals.

FIG. 12 shows the MEF were transiently transfected with GFP-LC3 and cultured in complete medium with or without OC (15μM) for 8 hours. The colocalization of GFP-LC3 and LysoTracker Red were analyzed by confocal microscopy. The enlarged images show the colocalization of two signals.

FIG. 13 shows HeLa or CNE cells treated with OC and cultured in either complete medium (DMEM with serum) or nutrient-deprived medium (EBSS without serum) for 24 hours. The cells were fixed and stained with propidium iodide (PI). OC (5μM) sensitizes nutrient-deprived cancer cells to apoptosis.

FIG. 14 shows the HeLa cells cultured in DMEM or EBSS medium treated with a certain amount of OC for 24 hours. Samples were analyzed by western blotting for cleaved Caspase 3, LC3 and SQSTM1. GAPDH was used as a loading control.

FIG. 15 shows the OC exhibits anticancer activity in cervical cancer xenograft.

Four weeks old nude mice were engrafted with HeLa cells and observed until tumors reached ˜100 mm³. Tumor-bearing mice were then treated vehicle, OC (12 μg) or Etopside (4 μg) (n=7) by intratumoral once every 2 days for a total of seven injections. Mice were killed and tumors resected and weighed 2 days after the final injection. Tumors from mice treated with OC and Etop were smaller than those of vehicle treated mice.

### DETAILED DESCRIPTION OF INVENTION

The present invention is not to be limited in scope by any of the specific embodiments described herein. The following embodiments are presented for exemplification only.

Macroautophagy (hereafter referred to autophagy for simplicity) is an evolutionarily conserved membrane process that results in the transporting of cellular contents to lysosomes for degradation. Autophagy involves the formation of double membrane vesicles, known as autophagosomes, which engulf intracellular contents such as mitochondria, endoplasmic reticulum and ribosomes, fuse with lysosomes for degradation. Autophagic degradation is an important regulator of cellular homeostasis as this process mediates the turnover of defective organelles, misfolded or aggregated proteins, and certain long-lived molecules. However, the role of autophagy extends beyond the general homeostatic removal, degradation and recycling of damaged proteins and organelles to many specific physiological and pathological processes such as tumourigenesis and cell death.

Microtubule-associated protein 1 light china 3 (LC3/MAP1LC3B), a homologue of yeast protein ATG8, serves as a marker protein for autophagosome. The change of autophagy process in live cells can be monitored by using a GFP-fused LC3 (GFP-LC3) protein. The number of GFP-LC3 puncta is very low under normal condition but rapidly increases when autophagy is activated by rapamycin or stress. However, the increase of GFP-LC3 level is not necessarily depended on autophagy induction. It could be the result of lysosome defect and associated with the inhibition of autophagy. To confirm the function of chemicals as either inducer or inhibitor of autophagy, more assay criteria such as monitoring autophagic flux is required. The poly-ubiquitin binding protein SQSTM1/p62 (also known as SQSTM1/sequestome 1) is selectively incorporated into autophagosomes through direct binding to LC3 and is efficiently degraded by autophagy, thus the total cellular expression levels of SQSTM1 correlate with autophagic activity. Therefore, by using several different concurrent methods to accurately assess the status and function of autophagic activity in any given biological setting, more specific agents will be developed to modulate autophagy and subsequently for use in anticancer therapy. In addition, novel autophagy regulators may also help to unravel the complex mechanisms in autophagy signaling pathways.

Compounds from natural plants or microbes are important resources for drugs against a wide variety of diseases such as cancer, malaria and infectious diseases. Many traditional Chinese medicines containing toxic compounds from plants exhibit antitumor effects and have been used for the different stages of cancer therapy.

Garcinia species (Family Guttiferae) are tropical evergreen trees and shrubs that are widely distributed in Southeastern Asia and their phytochemistry has been widely studied. Classic and caged xanthones have been isolated from various parts of these plants, and identified as their major bioactive components. Traditionally, Garcinia extract (called gamboge) has been used in folk and Chinese medicine to promote detoxification, and treat inflammation and wounds, and recently xanthones isolated from various Garcinia species also showed antibacterial, antioxidant, antiviral and neuroprotective effects.

In the last decade, most of the research on Garcinia species has focused on the anticancer activity of gambogic acid (GA), a caged xanthone found at high concentrations in gamboge as reported in Han QB, Xu HX (2009) Caged Garcinia xanthones: development since 1937. Curr Med Chem 16: 3775-3796. GA has been involved in the injectable antitumor drug since the 1970s. In 2004, GA has been granted permission for testing in clinical trials as a wide spectrum antitumor drug. GA and its derivatives are cytotoxic in many cancer cell lines by binding to the transferrin receptor and induction of G₂/M cell cycle arrest and mitochondrial and death receptor-mediated apoptosis. GA also reduces invasion and angiogenesis, telomerase mRNA expression and activity and tumor volume in vivo. However, the antitumor effect of GA is not selective and it induces toxicity to the liver and kidney, which limits its development into a clinically useful anticancer drug.

Due to the toxicity of GA, our recent research has focused on the discovery of novel and more selective compounds isolated from various Garcinia species. We have screened various components using different cancer cells, and found that several polyprenylated acylphloroglucinol (PPAP) compounds had potent cytotoxic effects on human colorectal cancer cell lines without affecting the normal human colon fibroblasts. According to our previous studies such as Feng C., et al., Int. J. Cancer: 2012, 131, 1445. Can and Huang S. X., et al., J. Nat. Prod. 2009, 72, 130-135, Oblongifolin C is the most potent PPAP compound. In this invention, we found that Oblongifolin C can significantly inhibit esophageal cancer cell (ESCC) ECA109 migration and invasion. Thus, the present invention provides Oblongifolin C for use as anticancer drug against high metastatic cancer. Furthermore, we also found that Oblongifolin C blocks both autophagic flux and lysosomal proteolytic activity. Oblongifolin C impairs autophagosome-lysosome fusion. Notably, Oblongifolin C efficiently sensitizes nutrient-deprived cancer cells to apoptosis in vitro. The anticancer activity of Oblongifolin C in cervical cancer xenograft mouse model in the present application, as seen by increased Casepse 3 cleavage, LC3 and SQSTM1 accumulation, and reduced expression of lysosomal cathepsins. Accordingly, the present invention provides Oblongifolin C for use as anticancer drug as autophagy inhibitor.

Extraction and Isolation

The air-dried and powdered pericarp (9.0 kg) of Garcinia yunnanensis Hu was extracted with acetone (20 L) at room temperature for three times. The extracted solution was evaporated under reduced pressure to yield a dark green residue (1.2 kg). The residue was chromatographed on silica gel eluted by CHCl₃, EtOAc, and acetone sequentially. The CHCl₃ fraction was evaporated in vacuum to give a residue (750 g), part of which (400 g) was subjected to silica gel column eluted with a gradient hexane/acetone system (100:0 to 0:100, v/v). Four fractions (I-V) were obtained on the basis of TLC analysis. Fraction I was purified by reversed-phase C-18 silica gel to afford Oblongifolin C (5.0 g).

Cell Culture

Human esophageal cancer cell line ECA109 were maintained in RPMI1640 (Gibco/Invitrogen) supplemented with 10% fetal bovine serum (Invitrogen), 100 U/ml penicillin-streptomycin (Gibco/ Invitrogen, 15140-122), within a humidified atmosphere containing 5% CO2 at 37° C.

HeLa, HCT116, CNE, MCF7, MDA-MB-231, HepG2 and MEFs were maintained in Dulbecco's modified Eagle's medium (Gibco/Invitrogen, 12800-017) supplemented with 10% fetal bovine serum 100U/m1 penicillin-streptomycin (Gibco/ Invitrogen, 15140-122) at 37° C. in a humidified 5% CO₂ incubator. For nutrient starvation, HeLa or CNE cells cultured in DMEM were washed three times with PBS, then cultured in Earle's Balanced Salt Solution (EBSS, Sigma, E6132) for indicated time point.

Anti-migration activity

Wound Healing Assay

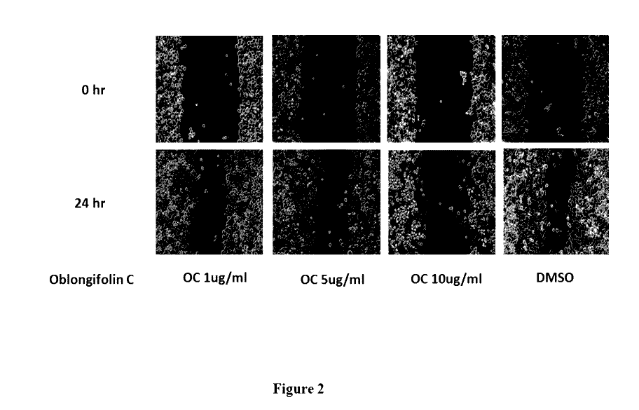

ECA109 cells were seeded on 12-well plates at a density of 1×10⁵ cells/well. After the cells reached sub-confluence, the mono-layer cells were wounded by scraping off the cells and then grown in medium for 24 hours. The migrated distance of cells was monitored and imaged under a microscope. As shown in FIG. 2, Oblongifolin C suppressed the wound healing in a concentration dependent manner.

Transwell Assay

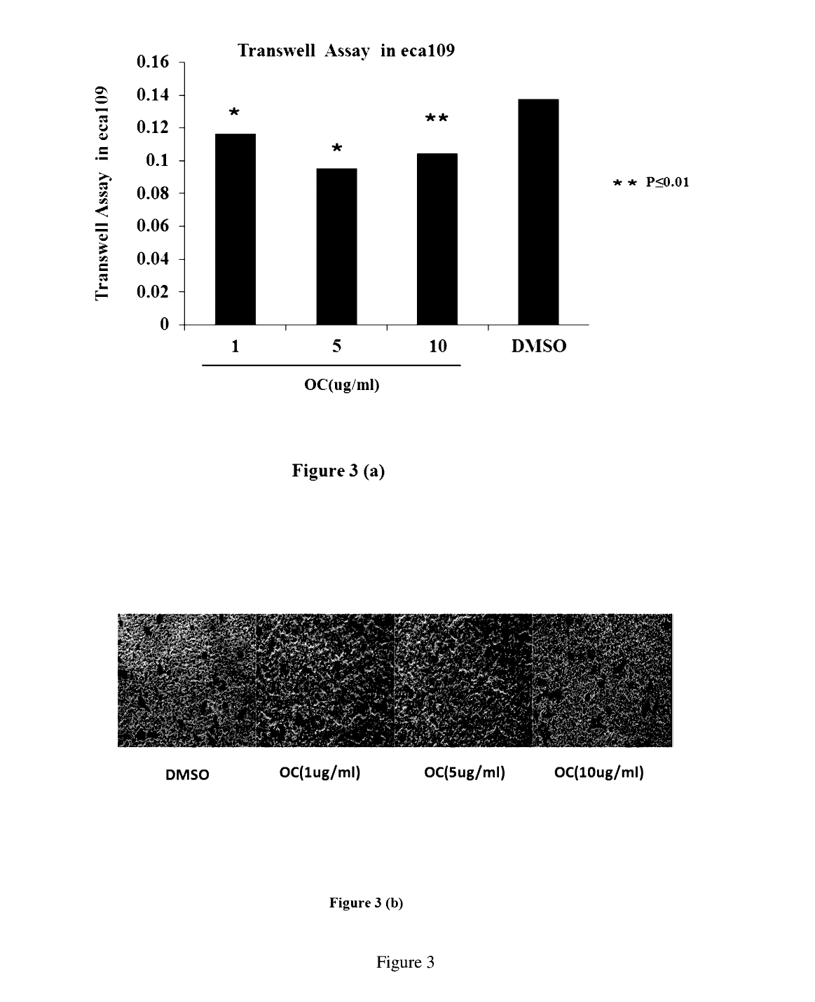

Cell migration was also determined using a transwell (Corning) with pore size of 8 μm. 5×10⁴ cells that were seeded in serum-free medium in the upper chamber, while medium containing 10% FBS in the lower chamber. After incubating for 24 hours at 37° C., cells in the upper chamber were carefully removed with a cotton swab and the cells that had traversed to reverse face of the membrane were fixed in methanol, stained with Giemsa, and counted. FIG. 3 showed the Oblongifolin C can suppress ECA109 cells migration through transwell at concentration dependent manner.

Anti-invasion Assay (Matrigel Assay)

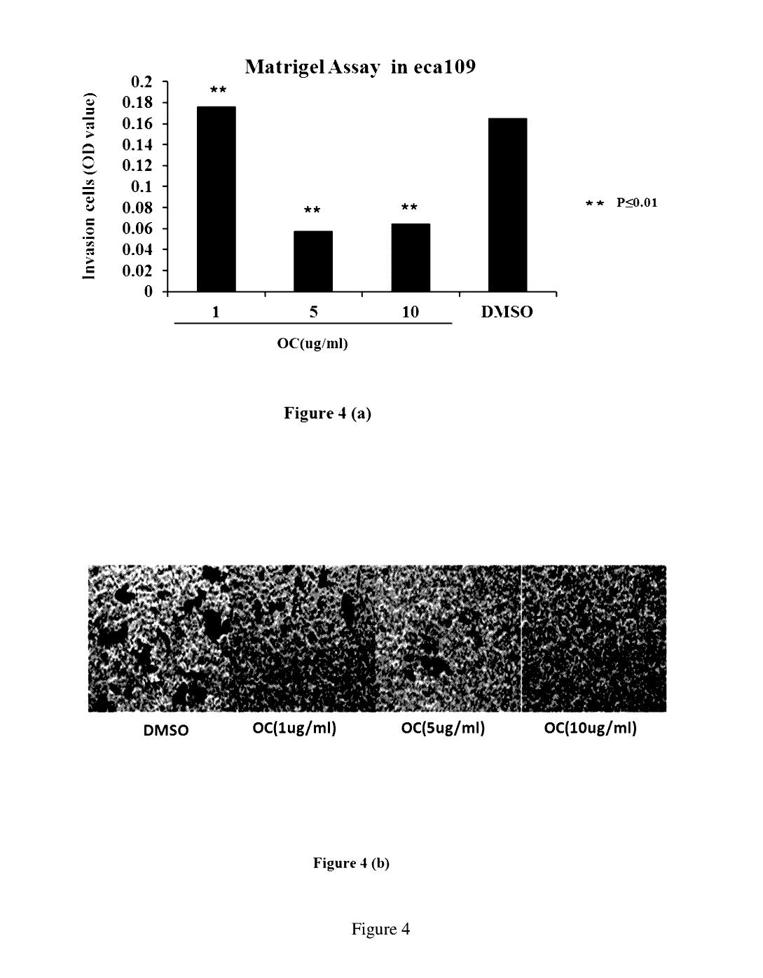

Cell invasion was determined using Matrigel (BD) coated transwell (Corning) with pore size of 8 μm. 5×10⁴ cells that were seeded in serum-free medium in the upper chamber, while medium containing 10% FBS in the lower chamber. After incubating for 72 hr at 37° C., cells in the upper chamber were carefully removed with a cotton swab and the cells that had traversed to reverse face of the membrane were fixed in methanol, stained with Giemsa, and counted. FIG. 4 showed the Oblongifolin C can suppress ECA109 cells invasion through matrigel at a concentration dependent manner.

Autophagy-related activity

GFP-LC3 translocation and quantitative analyses.

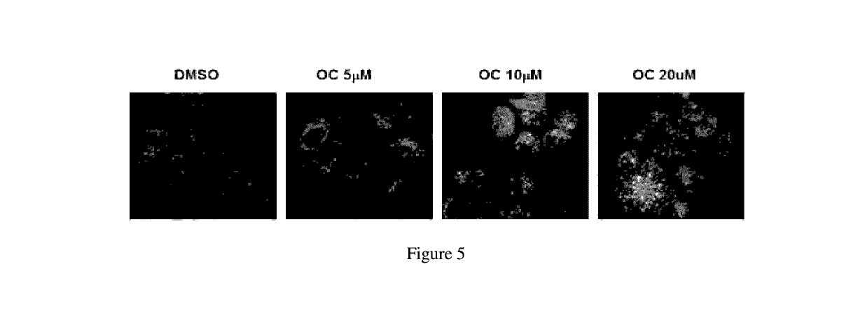

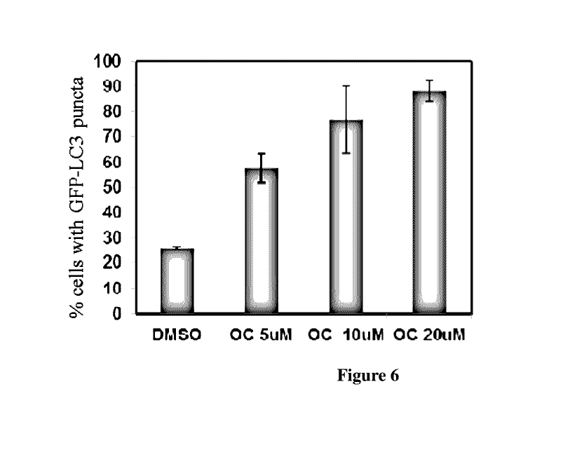

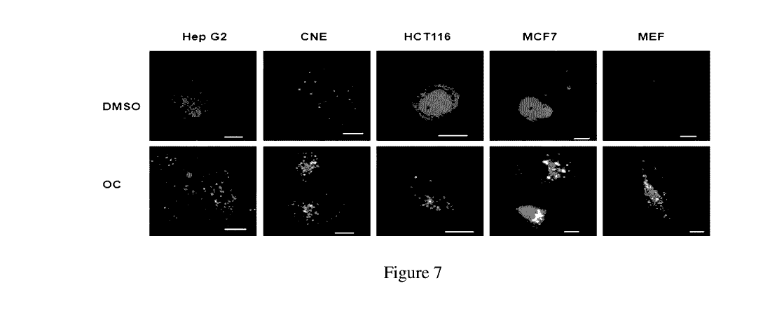

MEF, HeLa, CNE, HCT116, MCF7 and HepG2 cells were transfected with pEGFP-LC3 plasmid using lipofectamine 2000 (Invitrogen, 11668-019). One day after transfection, cells were treated with 10 μM Oblongifolin C for 24 hours prior to fixation. Image acquisition was done using an Olympus FV1000 confocal microscope. The number of GFP-LC3 dots was counted from at least 150 cells from randomly placed positions within each sample. FIG. 5 and FIG. 6 showed that Oblongifolin C could regulate GFP-LC3 puncta formation at concentration dependent manner. FIG. 7 showed that Oblongifolin C could regulate GFP-LC3 puncta formation in human hepatocytes (HepG2), human nasopharyngeal cancer cells (CNE), human colon cancer cells (HCT116), human breast cancer cells (MCF7) and mouse embryonic fibroblast cells (MEF).

Western blot

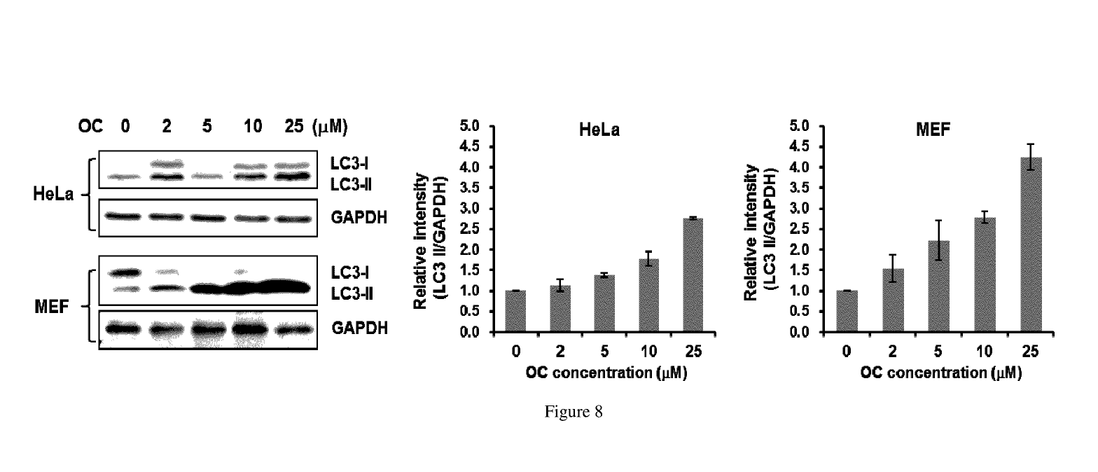

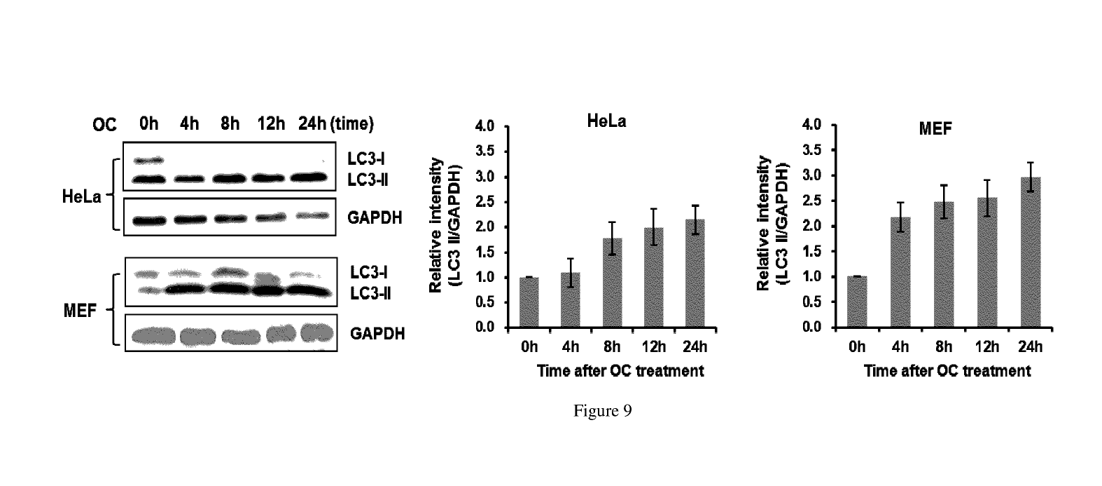

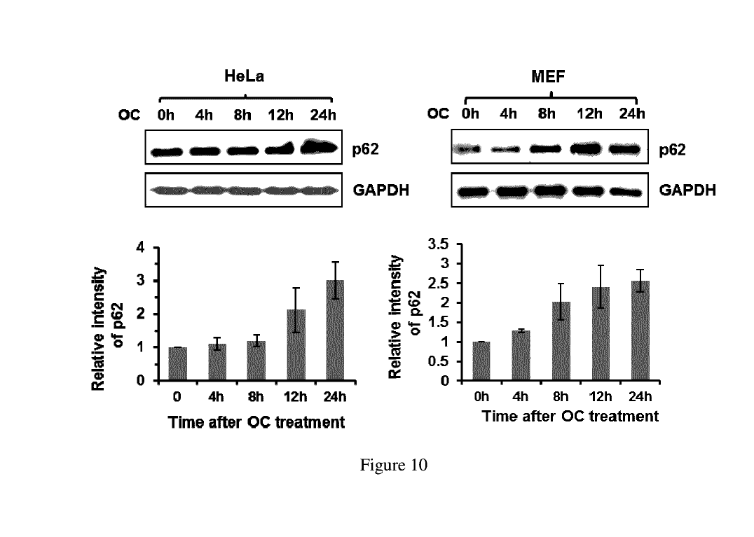

Cells were lysed in ice-cold whole cell extract buffer (50 mM pH8.0 Tris-HCl, 4M urea and 1% TritonX-100), supplemented with complete protease inhibitor mixture (Roche Diagnostics, 04693132001). Cell extracts were resolved by SDS-PAGE gel electrophoresis and transferred to a nitrocellulose membrane. After blocking with 5% non-fat milk in Tris-buffered saline containing 0.2% Tween-20, the membranes were probed with the following antibodies: LC3B (sigma, L7543), SQSTM1/p62 (MBL, PM045), GAPDH (Proteintech, 10494-1-AP), CASP3 (Cell signaling, #9962). Following incubation with horseradish peroxidase coupled secondary anti-mouse (KPL, 074-1806) or anti-rabbit antibodies (KPL, 474-1506), protein bands were visualized using ECL Blotting Detection Reagents (KPL, 54-61-00). As shown in FIG. 8, Oblongifolin C could increase the amount of LC3-II protein in a dose-dependent manner. FIG. 9 shows that Oblongifolin C could increase the amount of LC3-II protein in a time-dependent manner. FIG. 10 shows that Oblongifolin C could increase the amount of LC3-II protein in a time-dependent manner, suggesting that Oblongifolin C inhibits autophagic flux.

Live Cell Imaging and Colocalization Analysis

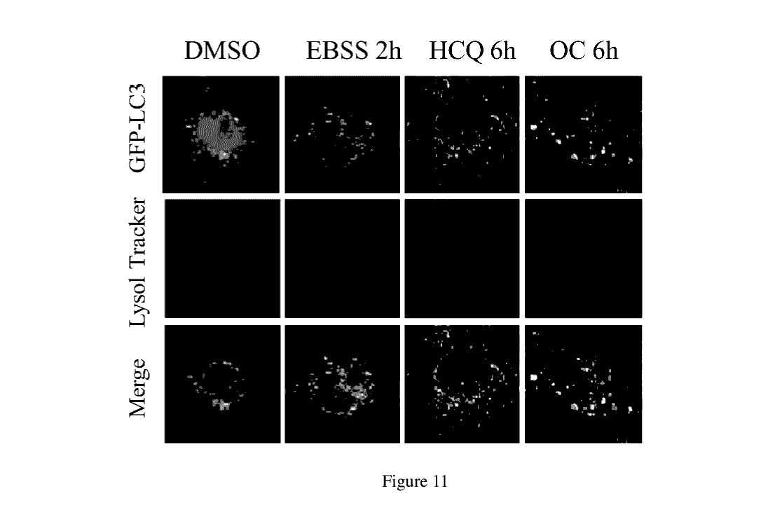

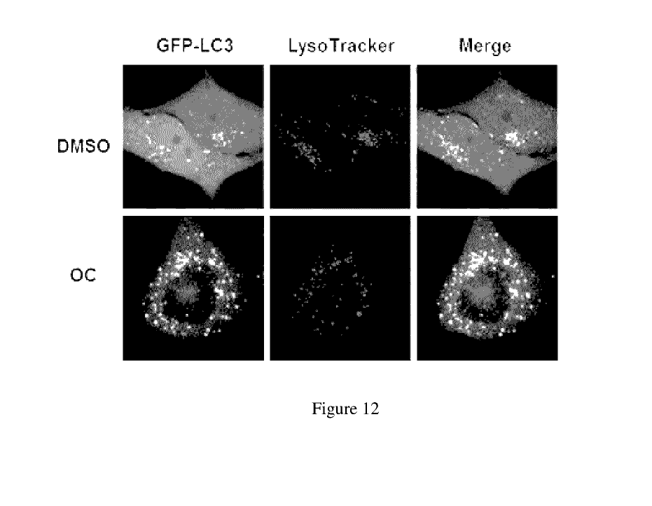

For lysotracker staining, HeLa or MEF cells grown on coverslips were stained with 50 nM Lysotracker Red DND-99 (Molecular Probes, Invitrogen, L7528) in pre-warmed medium for 20 min at 37° C. All of the samples were examined under an Olympus FV1000 confocal microscope (Olympus) equipped with a 63×oil immersion objective. The confocal images were acquired using FV10-ASW 2.0 software. Calculation of Pearson's correlation coefficient (PCC) was applied to quantify colocalization. PCC was calculated by FV10-ASW 2.0 software between the stack of images from two channels. As shown in FIG. 11, Oblongifolin C could block autophagosome-lysosome fusion as hydroxychloroquine sulfate (HCQ) in HeLa cells. Oblongifolin C also could block autophagosome-lysosome fusion in MEF (FIG. 12).

Flow cytometry

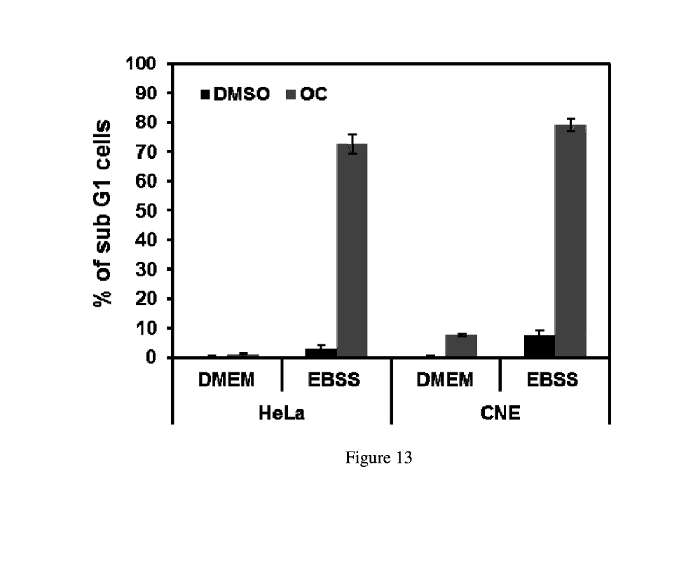

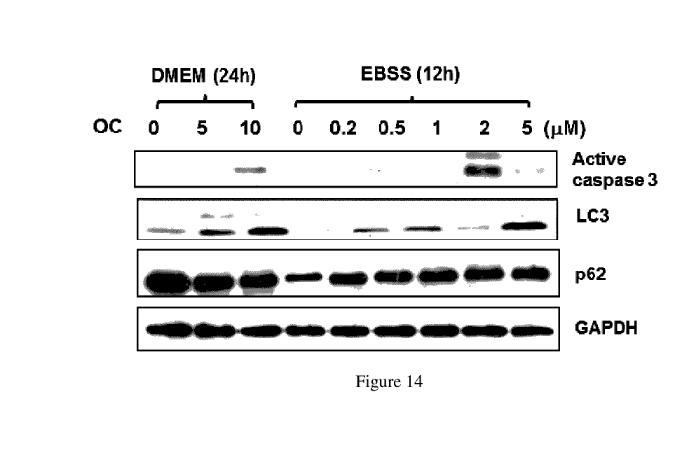

Cells were treated with Oblongifolin C then cultured in complete (DMEM with serum) or nutrient deprived medium (EBSS) for 24 hours. The cells were fixed in 70% ethanol in PBS overnight. For cell cycle distribution, cells were counterstained with propidium iodide and analyzed for DNA content by use of a BD Influx™ flow cytometer. As shown in FIG. 13, Oblongifolin C sensitizes nutrient-deprived cancer cells to apoptosis. As shown in FIG. 14, Oblongifolin C treatment resulted in Caspase 3 and cleavage in concentration-dependent manner in both complete (DMEM) and nutrient-deprived medium (EBSS), however, starvation dramatically increased the susceptibility of cancer cells to OC treatment. These data indicate that OC can eliminate the tolerance of cancer cells to nutrient starvation through Caspase 3-dependent apoptotic pathway.

Anti-Cancer Activity in Vivo

Four-week-old BALB/c nude mice were purchased from the Experimental Animal

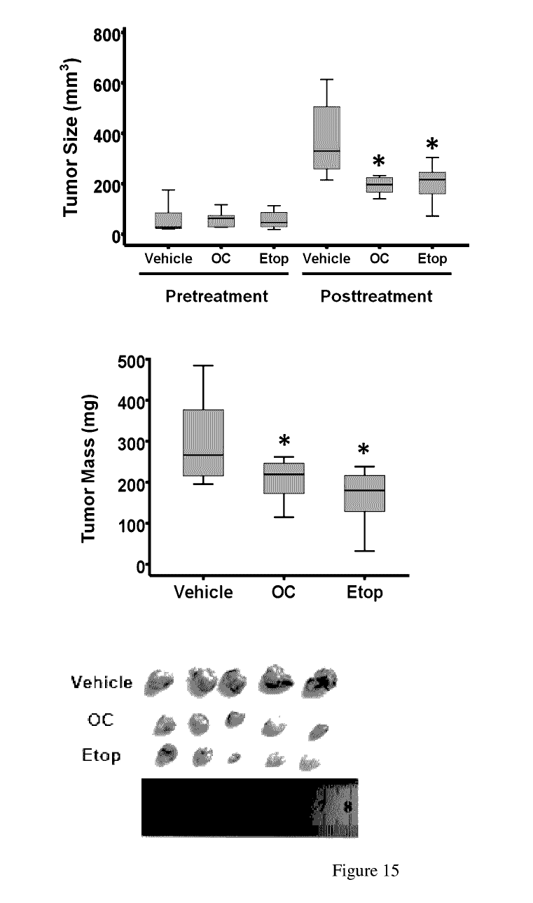

Center of Chinese Academy of Science (Shanghai, China) and kept in pathogen free environment at Experimental Animal Center in Shanghai University of Traditional Chinese Medicine. The mice have an average weight of 20 g. Approximately 2×10⁶ HeLa cells were injected into the left and right sides of the animals. Two weeks later, 15 mice bearing tumors around 100 mm³ in volume were randomly divided into vehicle control, etoposide (positive control) and oblongifolin C-treated groups. The mice were administered via intratumoral injection at the dose of 4 μg etopside or 12 μg OC in 200 μl solvent (0.05% DMSO, 0.05% Tween-80 in PBS) once every two days for two weeks. The tumor size was measured over a period of two weeks. As shown in FIG. 15, Oblongifolin C is shown to exhibit anticancer effect similar to etopside in cervical cancer xenograft.

Plant Material

The pericarp of Garcinia yunnanensis Hu were collected in Luxi of Dehong prefecture, Yunnan province, China in 2006. The plant material was identified by Dr. Chunfeng Qiao.

A herbarium sample was deposited in the Shanghai University of Traditional Chinese Medicine.

Discussion

In this invention, the anti-migration and anti-invasion effects of Oblongifolin C, a natural compound isolated from Garcinia yunnanensis Hu, are shown. The present application also demonstrates the anti-cancer and autophagic flux inhibition effects of Oblongifolin C, a natural compound isolated from Garcinia yunnanesis Hu. Oblongifolin C inhibits autophagic flux and blocks autophagosome-lysosome fusion. Furthermore, Oblongifolin C eliminates the tolerance of cancer cells to nutrient starvation and exhibits anticancer activity in cervical cancer xenograft. Oblongifolin C is developed as anticancer drug. In accordance with the present invention, the effective dosage of Oblongifolin C for mouse is 0.6 mg/kg (based on 12 μg of Oblongifolin C given to 20 g mouse). According to the dose translation formula, human equivalent dosage (mg/kg)=Animal dose (mg/kg) multiplied by animal Km/human Km, where mouse Km is 3 and human Km is 37 (Reagan-Shaw et al., Dose translation from animal to human studies revisited. FASEB J. 2008; 22(3):659-61.), the effective human equivalent dosage of Oblongifolin C of the present invention is 0.486 mg/kg, that is 2.43 mg per 50 kg of human.

In this invention, Oblongifolin C is a novel autophagic flux inhibitor and potent anticancer agent that selectively eliminates cancer cells under nutrient starvation. Thus, Oblongifolin C can be developed as anticancer drug and as an autophagy inhibitor.

### INDUSTRIAL APPLICABILITY

The present invention discloses a chemical entity isolated from natural sources for its therapeutic and medical uses. More particularly, it relates to compound that is naturally occurring in the plant of Garcinia yunnanensis Hu and its biological activity of antitumor and autophagic flux inhibition effects.

If desired, the different functions discussed herein may be performed in a different order and/or concurrently with each other. Furthermore, if desired, one or more of the above-described functions may be optional or may be combined.

While the foregoing invention has been described with respect to various embodiments and examples, it is understood that other embodiments are within the scope of the present invention as expressed in the following claims and their equivalents. Moreover, the above specific examples are to be construed as merely illustrative, and not limitative of the reminder of the disclosure in any way whatsoever. Without further elaboration, it is believed that one skilled in the art can, based on the description herein, utilize the present invention to its fullest extend. All publications recited herein are hereby incorporated by reference in their entirety.

What we claim:

1. A method of treating cancer comprising administering to a subject in need thereof a composition comprises an effective amount of compound of 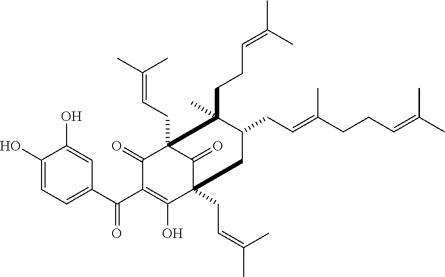 [SMILES: CC(C)=CCC/C(C)=C/C[C@@H]1C[C@]2(CC=C(C)C)C(=O)[C@@](CC=C(C)C)(C(=O)C(C(=O)c3ccc(O)c(O)c3)=C2O)[C@@]1(C)CCC=C(C)C]
wherein the effective amount is approximately 0.486 mg of said compound per kg of the subject.

2. The method of claim 1, wherein the cancer is cervical or esophageal cancer.

3. The method of claim 1, wherein the subject is a human.

4. The method of claim 1, wherein the composition is administered intratumorally.

5. The method of claim 1, wherein the composition is administered once every two days for two weeks.

6. The method of claim 1, wherein the effective amount is approximately 2.43 mg of said compound per 50 kg of the subject.
